(19) United States
(12) Patent Application Publication
(10) Pub. No.: US 2006/0246028 A1
(43) Pub. Date: Nov. 2, 2006

(54) Di-glycosil glyceryl compounds for the stabilisation and preservation of biomaterials

(76) Inventors: Helena Santos, Oeiras (PT); Pedro Miguel Lamosa Antonio, Lisboa (PT); Carla Jorge, Almada (PT); Milton Simoes da Costa, Oliveira do Bairro (PT)

Correspondence Address: RYNDAK & SURI LLP, 200 W. MADISON STREET, SUITE 2100, CHICAGO, IL, 60606, US

(21) Appl. No.: 10/553,992

(22) PCT Filed: Apr. 22, 2003
(86) PCT No.: PCT/PT03/00006

§ 371 Date: Jun. 27, 2006

(51) Int. Cl. A61K 8/73; A01N 1/02; C12Q 1/68; A61K 9/127; C11D 3/386; C08B 37/00

(52) U.S. Cl. 424/70.13; 435/2; 435/6; 424/450; 510/320; 536/123

(57) ABSTRACT

The present invention consists in the utilization of glycosyl(1-2)glycosyl(1-2)glyceryls, like mannosyl(1-2)glucosyl(1-2)glycerate, or mixtures thereof alone or as constituents in a suitable formulation as protectors and/or stabilisers of enzymes, proteins, antibodies, DNA or RNA molecules, biological membranes, lipossomes, lipid related substances or other cellular components and biomaterial against general stress.

### FIELD OF THE INVENTION

The present invention concerns the use of di-glycosyl glyceryl compounds alone or as constituents of formulations to protect and/or stabilise enzymes or other cellular components and biomaterials against general stress, namely caused by heat, high osmolarity, free-radicals, desiccation, freeze-drying, and repetitive use.

Said compounds obey to the general formula depicted in FIG. 1 and have the general name of glycosyl(1-2)glycosyl(1-2)glyceryls.

### DESCRIPTION OF THE INVENTION AND STATE OF THE ART

The accumulation of low-molecular mass organic solutes such as, trehalose, polyols or ectoines, is a prerequisite for osmotic adjustment of many mesophilic microorganisms. However, very unusual solutes namely, di-myoinositol-phosphate, di-mannosyl-di-myo-inositol-phosphate, diglycerol phosphate, mannosylglycerate, and mannosylglyceramide, have been identified in thermophilic and hyperthermophilic microorganisms and the intracellular content of these solutes increases in response to stress conditions, such as high osmolarity or high temperature.

Mannosylglycerate and diglycerol phosphate have been studied to a greater extent and have been shown to protect enzymes and proteins in vitro better than compatible solutes from mesophiles [1-3]. Moreover, the application of compatible solutes from thermophilic or hyperthermophilic organisms as stabilising agents of biomaterials has been disclosed in several patent applications [4-6].

We have discovered a novel compatible solute in the thermophilic bacterium Petrotoga miotherma, an organism that grows optimally at 55° C., but is able to grow as high as 65° C. When subjected to salt stress this organism accumulates large amounts (above 1 μmol/mg of protein) of a novel di-sugar compound. After extraction, purification, and full spectroscopic characterization by Nuclear Magnetic Resonance, we have determined the molecular structure of this, to date, unknown compound as α((1-2))mannopyranosyl-α((1-2))glucopyranosyl-glycerate.

It is interesting to note that the molecular structure of this compatible solute comprises the two moieties (mannosyl and glyceryl) present in mannosylglycerate, a solute widely distributed among thermophiles and hyperthermophiles [7]. In addition, there is a glucosyl moiety linking the mannosyl and glyceryl moieties.

Mannosylglycerate is a well-known biostabiliser of thermophilic origin, whose industrial application is protected under a European patent application [4]. The thermophilic origin of the novel solute combined with the structural resemblance to mannosylglycerate leads us to propose that this novel solute has stabilising properties as good or superior to those already demonstrated for mannosylglycerate. In this respect, it will serve as a stabiliser in various commercial, industrial, medical, pharmaceutical, diagnostic, cosmetic, or academic research applications.

The enhanced protein stability rendered by certain low-molecular mass organic solutes allows enzymes to function under more severe conditions of temperature, pressure, ionic strength, pH, presence of detergents or organic solvents. One of the priorities of modern biotechnology is to obtain stable enzymes or agents that stabilise those enzymes against thermal or chemical denaturation. The ability of some compatible solutes to stabilise enzymes is, therefore, of great importance to modern biotechnology. This point is obviously extended to all proteins that are used or can be used in processes where their stability is an issue, since all proteins either with or without enzymatic activity share the same overall basic elements of structure and may be protected against denaturation or inactivation through the same general mechanisms or processes.

It must also be stressed that compatible solutes protect proteins, cell membranes, lipossomes, and cells from the deleterious effects of desiccation, and possess strong moistening properties. The preservation of desiccated or lyophilized cell components and biomaterials has many applications in medicine, pharmaceutical industry, cosmetic industry, food industry, and scientific research. In spite of the great importance of desiccation and freezing in the conservation of biological samples, denaturation of proteins or a decrease of the viable count of cultures inevitably takes place during utilization, and could be prevented or diminished by the use of low molecular mass stabilisers.

Also, the stability of nucleic acid molecules, like DNA, or RNA, can be improved by the addition of compatible solutes from hyperthermophiles, as described for ectoines [8], and their use in several applications in medicine, pharmaceutical industry, or scientific research can be envisioned.

### BRIEF DESCRIPTION OF THE DRAWING

FIG. 1.

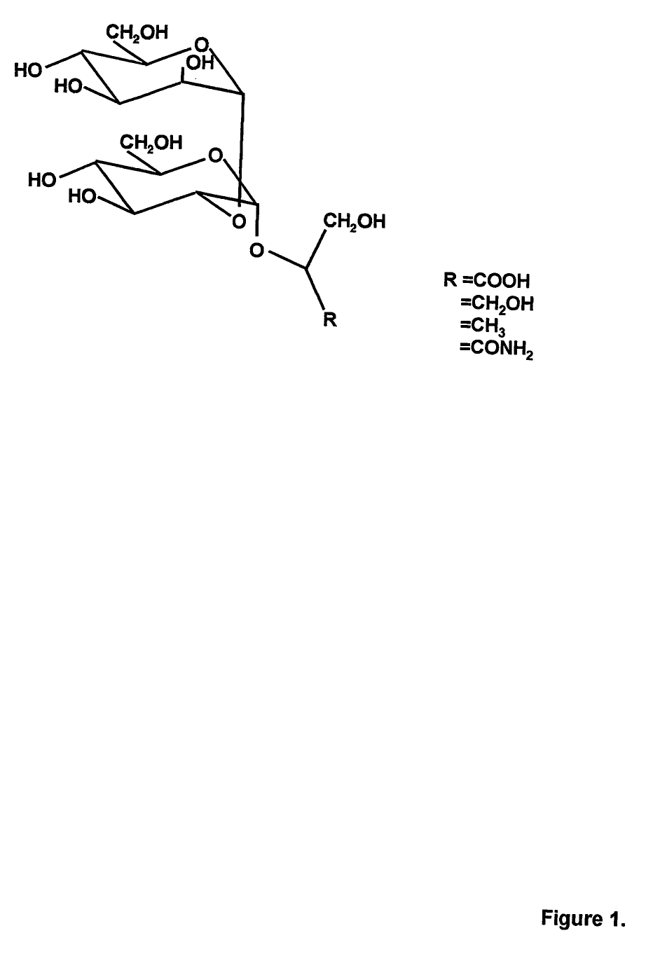

Depicts the generic chemical structure of glycosyl(1-2)glycosyl(1-2)glyceryls in all its possible stereoisomeric forms. The figure is intended to represent all hexoses either in the α or in the β configuration. The letter “R” is intended to represent a carboxylate, an amide, a primary alcohol group, or a methyl group.

### REFERENCES

[1] Ramos, A., N. D. H. Raven, R. J. Sharp, S. Bartolucci, M. Rossi, R. Cannio, J. Lebbink, J. van der Oost, W. M. de Vos and H. Santos. 1997. Stabilisation of enzymes against thermal stress and freeze-drying by mannosylglycerate. Appl. Environ. Microbiol. 63:4020-4025.
[2] Borges, N., A. Ramos, N. D. H. Raven, R. J. Sharp, and H. Santos. 2002. Comparative study of the thermostabilising properties of mannosylglycerate and other compatible solutes on model enzymes. Extremophiles 6:209-216.
[3] Lamosa, P., A Burke, R. Peist, R. Huber, M. Liu, G. Silva, C. Rodrigues-Pousada, J. LeGall, C. Maycock, and H. Santos. 2000. Thermostabilisation of proteins by diglycerol phosphate, a new compatible solute from the hyperthermophile Archaeoglobus fulgidus. Appl. Environ. Microbiol. 66:1974-1979.
[4] Santos, H., A Ramos, M. S. da Costa. Thermostabilisation, osmoprotection, and protection against desiccation of enzymes, cell components and cells by mannosylglycerate. European Patent no. 97670002.1
[5] Santos, H., P. Lamosa, C. Maycock and A Burke. 1999. Thermostabilisation, osmoprotection, and protection against desiccation of enzymes, cell components and cells by di-glycerol-phosphate. European Patent no. 98670002.9
[6] Santos, H., L. O. Martins, L. S. Carreto, and M. S. da Costa. 1996. Utilização de fosfato de di-manosil-di-mio-inositol e de fosfato de 1,3′-di-mio-inositol na termo-estabilisação, osmoproteção e protecção contra a desidratação de componentes celulares e células. Portuguese patent no. 101813.
[7] Santos. H., and M. S. da Costa. 2001. Organic solutes from thermophiles and hyperthermophiles. Methods Enzymol. 334:302-315.
[8] Malin G, Iakobashvili R, and Lapidot A (1999) Effect of tetrahydropyrimidine derivatives on protein-nucleic acids interaction. Type II restriction endonucleases as a model system. J. Biol. Chem. 274:6920-6929.

1. The utilization of glycosyl(1-2)glycosyl(1-2)glyceryls, namely mannosyl(1-2)glucosyl(1-2)glycerate, wherein the compound is in any of its possible stereoisomeric forms, alone or as a constituent in a suitable formulation to protect and/or stabilize enzymes or other cellular components and biomaterials against general stress.

2. The utilization of glycosyl(1-2)glycosyl(1-2)glyceryls according to claim 1 wherein the mannosyl residue is replaced by any six member ring carbohydrate residue, namely glucose, galactose, gulose, talose, fucose, rhamnose, idose, or altrose.

3. The utilization of glycosyl(1-2)glycosyl(1-2)glyceryls according to claim 1 wherein the glucosyl residue is replaced by any six member ring carbohydrate residue, namely mannose, galatose, gulose, talose, fucose, rhamnose, idose, or altrose.

4. The utilization of glycosyl(1-2)glycosyl(1-2)glyceryls according to claim 1 wherein the glycerate residue is replaced by glycerol.

5. The utilization of glycosyl(1-2)glycosyl(1-2)glyceryls according to claim 1 wherein the glycerate residue is replaced by glyceramide.

6. The utilization of glycosyl(1-2)glycosyl(1-2)glyceryls according to claim 1 wherein the glyceryl residue is replaced by a actyl residue.

7. The utilization of glycosyl(1-2)glycosyl(1-2)glyceryls according to claim 1 wherein the glyceryl residue is replaced by propylene glycol.

8. The utilization of glycosyl(1-2)glycosyl(1-2)glyceryls according to claim 1 wherein the glycosyl residues are in the α or β configuration.

9. The utilization of glycosyl(1-2)glycosyl(1-2)glyceryls according to claim 1 wherein the glyceryl residue in in the D or L configuration.

10. The utilization of glycosyl(1-2)glycosyl(1-2)glyceryls according to claim 1 wherein to protect enzymes or other proteins against temperature denaturation induced by purification, transport and/or storage.

11. The utilization of glycosyl(1-2)glycosyl(1-2)glyceryls or mixtures thereof according to claim 1 wherein as a protector of the activity of polymerase chain reaction (PCR) enzymes for clinical, biological and industrial purposes during the storage, as well as the high-temperature recycling of the enzymes.

12. The utilization of glycosyl(1-2)glycosyl(1-2)glyceryls or mixtures thereof according to claim 1 wherein as a stabilizer or enzymes or other proteins during lyophilization, desiccation or freeze-drying and storage at low temperatures.

13. The utilization of glycosyl(1-2)glycosyl(1-2)glyceryls or mixtures thereof according to claim 1 wherein as a stabilizing agent during the manufacture, storage and assays using test kit enzymes, for diagnostic, biological and industrial purposes.

14. The utilization of glycosyl(1-2)glycosyl(1-2)glyceryls or mixtures thereof according to claim 1 wherein to stabilize enzymes or other proteins during their routine utilization for clinical, biological and industrial purposes.

15. The utilization of glycosyl(1-2)glycosyl(1-2)glyceryls or mixtures thereof according to claim 1 wherein for the protection or stabilization of antibodies for clinical, biological, research, and industrial purposes.

16. The utilization of glycosyl(1-2)glycosyl(1-2)glyceryls or mixtures thereof according to claim 1 wherein for the protection or stabilization of vaccines of proteic or non-proteic nature for clinical, or industrial purposes.

17. The utilization of glycosyl(1-2)glycosyl(1-2)glyceryls or mixtures thereof according to claim 1 wherein for the protection of biomaterials against stress such as desiccation and lyophilization of cell membranes, liposomes, liposome-containing cosmetics or lipid related substances.

18. The utilization of glycosyl(1-2)glycosyl(1-2)glyceryls or mixtures thereof according to claim 1 wherein for the structural protection or stabilization of DNA or RNA molecules.

19. The utilization of glycosyl(1-2)glycosyl(1-2)glyceryls or mixtures thereof according to claim 1 wherein for the protection, stabilization or improved performance of DNA or RNA microarrays.

20. The utilization of glycosyl(1-2)glycosyl(1-2)glyceryls or mixtures thereof according to claim 1 wherein as an additive to cosmetics to improve moistening properties stabilize compounds or liposomes or as a suppressor of free radicals.

21. The utilization of glycosyl(1-2)glycosyl(1-2)glyceryls or mixtures thereof according to claim 1 wherein for protection against damage caused by lyophilization, desiccation, high temperatures, and freezing to microbial cells.

22. The utilization of glycosyl(1-2)glycosyl(1-2)glyceryls or mixtures thereof according to claim 1 wherein the compound is a constituent in a suitable formulation to protect and/or stabilize enzymes or other cellular components and biomaterials.
